(19) United States
(12) Patent Application Publication
(10) Pub. No.: US 2008/0274206 A1
(43) Pub. Date: Nov. 6, 2008

(54) Stabilised Oxygen Releasing Composition

(75) Inventors: Changoer Lekhram, Ijsselstein (NL); Willem Frederik Van Den Bosch, Oegstgeest (NL); Hendrik Jan Cornelis Meijerink, Brussel (BE)

Correspondence Address: FOLEY AND LARDNER LLP;SUITE 500, 3000 K STREET NW, WASHINGTON, DC, 20007, US

(73) Assignee: Ngen Pharmaceuticals N.V.

(21) Appl. No.: 11/576,880

(22) PCT Filed: Jul. 10, 2005
(86) PCT No.: PCT/NL05/00728

§ 371 Date: Mar. 14, 2008

Related U.S. Application Data
(60) Provisional application No. 60/616,229, filed on Oct. 7, 2004.

(51) Int. Cl. A61K 33/40

(52) U.S. Cl. 424/613

(57) ABSTRACT

The present invention relates to stabilised liquid oxygen releasing composition comprising a component (a) selected from the salts consisting of cations Aₙ⁺ and anions derived from halogen oxides according to the general formula [OₘX]⁻ wherein A is a metal selected from Groups 1 or 2 of the Periodic System of the Elements, X is a halogen atom, m=1-4, n=1 or 2, a component (b) selected from the group of oxygen donors, a component (c) selected from the group of oxygen donor stabilising agents and a component (d) selected from the group of liquid binders. The stabilised liquid oxygen releasing composition can be used in pharmaceutical compositions, cosmetic compositions and food applications, e.g. skin care products, dental care products, tooth whitening products including chewing gums.

### SUMMARY OF THE INVENTION

The present invention relates to a stabilised liquid oxygen releasing composition, said stabilised liquid oxygen releasing composition being in particular suitable for pharmaceutical, cosmetic and food applications.

### OBJECT OF THE INVENTION

The object of the invention is to provide a stabilised liquid oxygen releasing composition, said composition comprising a component (a) selected from the salts consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2, a component (b) selected from the group of oxygen donors, a component (c) selected from the group of oxygen donor stabilising agents and a component (d) selected from the group of liquid binders.

According to the invention, the liquid composition may be a product having viscosity properties and flow characteristics that are comparable with those of rather thick to highly viscous, optionally thixotropic, liquids such as gels, pastes, suspensions, dispersions or emulsions. The liquid composition may have visco-elastic properties as well. Examples of such liquids are peanut butter, toothpaste, ointments, creams, shampoos and the like. The liquid composition may also be of low viscosity such as aqueous solutions, emulsions and dispersions, which are easy to pour. As a consequence, the liquid composition according to the present invention is not intended to be a composition consisting essentially or exclusively of solid materials, e.g. a mixture of powders.

Compositions comprising component (a) and component (b) are for example disclosed in U.S. Pat. No. 6,017,515 which is incorporated by reference herein. According to U.S. Pat. No. 6,017,515, the preferred component (a) is sodium hypochlorite and the preferred component (b) is sodium perborate tetrahydrate. The disadvantage of the compositions disclosed in U.S. Pat. No. 6,017,515 is that these compositions are rather unstable, i.e. that they release oxygen at a relatively high rate and in an uncontrolled manner with the consequence that the activity of the compositions decreases rapidly during use or application and when stored for prolonged periods. U.S. Pat. No. 6,017,515 therefore proposes either to keep components (a) and (b) separate just before use or, when already combined, to store the compositions at low temperature. It is evident to the person skilled in the art that uncontrolled release of the active component, i.e. oxygen, by pharmaceutical or cosmetic compositions is not desired since the composition looses its activity very rapidly and does not provide a long lasting effect. In addition, the compositions disclosed in U.S. Pat. No. 6,017,515 are unstable (unless components (a) and (b) are kept separate) which necessitates special conditions to preserve the activity of the compositions during storage and transportation up till the moment the composition is used by the customer, e.g. a physician or a patient. Finally, if components (a) and (b) are kept separate, they must first be combined prior to the application of the composition. When the composition is intended for example in pharmaceutical applications, this usually requires experienced and qualified personnel. Alternatively, when the composition is intended for example in cosmetic applications, special and complex packaging of the component is required to enable safe use of the composition by the customer. It is therefore apparent to the person skilled in the art that it is desired to have a composition comprising components (a) and (b) which releases the active component in a controlled manner and which shows a long lasting effect. Moreover, it is apparent that it is highly desirable to have such a composition comprising components (a) and (b) that is stable during storage and transportation under normal conditions and that is safe to use by the end user.

A solid composition consisting essentially of sodium perborate monohydrate, lithium hypochlorite and phosphate salts is disclosed in for example U.S. Pat. No. 3,793,211 which is incorporated by reference herein. This composition is only stable in the solid state as upon contact with water the composition rapidly decomposes.

EP A 0.085.891, incorporated by reference herein, discloses a composition for the cleaning and hygiene of the teeth which comprises a suspension of water soluble, non-abrasive salts selected from a large range of compounds which includes potassium perchlorate and sodium perborate. EP A 0.085.891 does, however, nowhere specifically disclose the combination of components (a) and (b).

GB A 552.803 discloses a solution that is prepared from borax (disodium tetraborate), calcium hypochlorite and other components, wherein the borax and the other components are added to water and subsequently boiled. In a next step the calcium hypochlorite is added and the mixture is allowed to cool while stirred. After a period of time, suspended matter has settled and a clear liquid and a paste is obtained which are separated by decantation. It is therefore clear to the person skilled in the art that the solution only contains reaction products of the original ingredients.

Other pharmaceutical and/or cosmetic compositions containing either component (a) or component (b) are also known in the art for a long time. Compositions containing component (a) are for example disclosed in GB A 1.469.398, GB A 2.289.841, GB A 2.290.233, WO 96/13245 and WO 98/04235 whereas compositions containing component (b) are for example disclosed in U.S. Pat. No. 3,574,824, U.S. Pat. No. 5,041,280, U.S. Pat. No. 5,264,205, U.S. Pat. No. 5,279,816, U.S. Pat. No. 5,302,375, U.S. Pat. No. 5,616,313, U.S. Pat. No. 5,632,972, U.S. Pat. No. 5,648,064, U.S. Pat. No. 5,858,332, U.S. Pat. No. 6,409,993 and U.S. Pat. No. 6,500,408, all incorporated by reference herein.

### SUMMARY OF THE INVENTION

The present invention relates to a stabilised liquid oxygen releasing composition, said composition comprising a component (a) selected from the salts consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2, a component (b) selected from the group of oxygen donors, a component (c) selected from the group of oxygen donor stabilising agents and a component (d) selected from the group of liquid binders.

### DETAILED DESCRIPTION OF THE INVENTION

The present invention is directed to a stabilised liquid oxygen releasing composition wherein the release of the active agent that is believed to be oxygen is controlled by an oxygen donor stabilising agent. The advantage of the invention is in particular that the composition is stable and has a long lasting effect. A further advantage of the invention is that the composition can be easily packaged and that the composition is safe to transport and to store. Another advantage of the present invention is that the composition can be used by the consumer in a safely and easy manner.

More particularly, the composition according to the present invention is directed to a stabilised liquid oxygen releasing composition, said composition comprising a component (a) selected from the salts consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2, a component (b) selected from the group of oxygen donors, a component (c) selected from the group of oxygen donor stabilising agents and a component (d) selected from the group of liquid binders.

In the formula [OₘX]⁻X can be fluorine, chlorine, bromine or iodine, preferably chlorine. Examples of the anion [OₘX]⁻ are the hypochlorite, hypoiodite, chlorite, iodite, chlorate, bromate, iodate, perchlorate and periodate anions. Since it is preferred that m=1, the anion [OₘX]⁻ is most preferably the hypochlorite anion.

Component (b) is preferably selected from oxygen donors selected from the group consisting of metal borate compounds, metal peroxide compounds, metal percarbonate compounds (also known as carbonate peroxyhydrate compounds), metal persulfate compounds (also known as peroxosulfur compounds), metal perphosphate compounds, wherein the metal is an alkaline or alkaline-earth metal, halogen oxide compounds, hydrogen peroxide, and organic peroxides. More preferably, component (b) is selected from the group consisting of metal perborate compounds (also known as peroxoborate compounds), metal percarbonate compounds, metal peroxide compounds, hydrogen peroxide, halogen oxide compounds and organic peroxides.

According to the invention, the halogen oxide compounds are preferably formed in situ from halogen oxide compound precursors. Suitable halogen oxide compound precursors are for example metal halite such as sodium chlorite and metal hypohalite such as sodium hypochlorite compounds as is well known in the art.

According to the invention, the perborate compounds are preferably selected from the salts consisting of cations Aₙ⁺ and anions derived from borates according to the general formula [BₚO_{q}]^{r-}, wherein A is a metal selected from Groups 1 or 2 of the Periodic System of the Elements, and p=1-4, q=1-8, and r=1-3. Examples of the anion [BₚO_{q}]^{r-} are perborate (BO₃)⁻, metaborate (BO₂)⁻, orthoborate (BO₃)³⁻, hypoborate (B₂O₄)₂₋ and pyroborate or tetraborate anions (B₄O₇)²⁻. Preferably, p=1, q=2 or 3 and r=1. Most preferably, p=1, q=3 and r=1 which implies that the most preferred anion is perborate. According to the invention, the percarbonate compounds are preferably selected from the salts consisting of cations Aₙ⁺ and anions CO₃²⁻. This implies that the percarbonates do not contain C—O—O-groups (the latter being known as peroxocarbonates). Additionally, the peroxosulfur compounds are preferable selected from peroxomonosulphates and peroxodisulphates. The metal peroxide compounds are preferably selected from alkaline-earth metal peroxide compounds, in particular calcium peroxide and magnesium peroxide. The organic peroxide is preferably carbamide peroxide. The halogen oxide is preferably chlorine dioxide ClO₂.

According to the invention, A is preferably selected from the group consisting of lithium, sodium, potassium, magnesium and calcium and is most preferably sodium. Consequently, component (a) is most preferably sodium hypochlorite and component (b) is most preferably sodium perborate.

Components (a) and (b) can contain one or more molecules of water as water of crystallisation. However, for component (a) preferably an aqueous solution thereof is used. Component (b) can also contain one or more molecules of water as water of crystallisation, for example the monohydrates, dihydrates, trihydrates and tetrahydrates. According to the invention, all hydrates of both components (a) and component (b) can be used. If component (a) or component (b) occur in different polymorphs, all these polymorphs can be used in the present invention.

For the sake of clarity, if component (b) is for example sodium perborate tetrahydrate, the compound Na₂BO₃.4H₂0 is intended which at present is more often defined as Na₂[B₂(O₂)₂(OH)₄].6H₂O; the common name of the later compound is sodium peroxoborate hexahydrate. Likewise, if component (b) is for example sodium perborate monohydrate, the compound Na₂BO₃.H₂0 is intended which at present is more often defined as Na₂[B₂(O₂)₂(OH)₄]. Reference is made to Kirk-Othmer, Encyclopedia of Chemical Technology, 4^{th} Edition, Volume 18, pages 202-229 (1996) for the nomenclature of such compounds.

According to the invention, oxygen donor stabilising agent (c) is preferably selected from the group consisting of organic acids or their (monovalent or polyvalent) pharmaceutically acceptable salts, preferably inorganic salts wherein the cations of the salts are preferably metals selected from Groups 1 or 2 of the Periodic System of the Elements, or from the group consisting of saccharides.

The organic acids are preferably selected from the group consisting of chelating organic acids. The chelating organic acids are preferably selected from carboxylic acids containing one or more hydroxy and/or amino groups and polycarboxylic acids optionally containing one or more hydroxy and/or amino groups.

Alternatively but depending on the intended use of the composition according to the invention, the chelating organic acids may be selected from polyphosphonic acids or their pharmaceutically acceptable salts as disclosed in U.S. Pat. No. 6,265,444 which is herein incorporated by reference, although then it is preferred that the polyphosphonic acid is used in combination with a carboxylic acid containing one or more hydroxy and/or amino groups or polycarboxylic acid optionally containing one or more hydroxy and/or amino groups such as EDTA. In this patent application a polyphosphonic acid is to be understood as a compound containing at least two —PO₃H₂ moieties or the pharmaceutical acceptable salt thereof. Preferably, the polyphosphonic acid has either formula I or formula II,

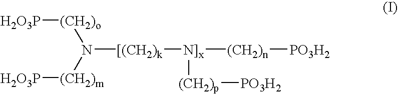

wherein x is an integer of 0 to 3 and k, m, n, o and p are each independently an integer of 1 to 4. Preferably, x is 2 and k, m, n, o and p are each 1 or 2;

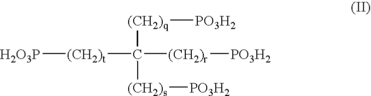

wherein q, r, s and t are each independently an integer of 0 to 4. Preferably q, r, s and t are 0 or 1, and most preferably all are zero.

The saccharides are preferably monosaccharides or disaccharides. Preferred examples are glucose, fructose, sucrose, maltitol and lactitol.

According to the invention, it is most preferred that the polycarboxylic acids contain one or more hydroxy and/or amino groups and such acids are generally known as hydroxypolycarboxylic acids and aminopolycarboxylic acids. Very suitable examples of the oxygen donor stabilising agents according to the invention are gluconic acid, ethylenediaminotetraacetic acid (EDTA), nitrilotriacetic acid (or 1,1′,1″-tricarboxytrimethylamine) and methylglycinediacetic acid or their salts. According to the present invention the most preferable oxygen donor stabilising agent (c) is sodium gluconate.

The amount of the oxygen donor stabilising agent (c) in the composition according to the invention is essential. According to the invention, the molar ratio of component (b) to component (c) is preferably 0.1-5.0 (b):1.0 (c). More preferably, this molar ratio is 0.3-3.0 (b):1.0 (c) and in particular 0.5-2.5 (b):1.0 (c). However, these ratios are dependent from the intended use of the composition.

Moreover, if the composition according to the invention is intended for the use in a dental care product, the molar ratio of component (b) to component (c) is in particular in the range of 0.5-0.9 (b):1.0 (c). However, if the composition according to the invention is intended for the use in a skin care product the molar ratio of component (b) to component (c) is in particular in the range of 1.0-3.0 (b):1.0 (c). On the other hand, in whitening gels or bleaching gels, it is preferred that the molar ratio of component (b) to component (c) is higher, i.e. 4.0-8.0 (b):1.0 (c), more preferably 4.5-7.5 (b):1.0 (c).

The ratios of components (a) and (b) is essential according to the invention. U.S. Pat. No. 6,017,515 discloses that the preparation disclosed therein can be prepared from a constituent (a) comprising 1-95% by weight, preferably 1-50% by weight of component (a) defined above and from a constituent (b) comprising 1-95% by weight, preferably 1-50% by weight of component (b) defined above, the preferred ratios of constituents (a) and (b) not being specified which implies that these ranges cover almost every possible molar ratio. Example II of U.S. Pat. No. 6,017,515, however, discloses a 1:1 weight ratio of constituents (a) and (b), wherein constituent (a) contains 5 ml 4% sodium hypochlorite in water (a preferred example of component (a) defined above) in water and constituent (b) contains 3 g sodium perborate tetrahydrate (a preferred example of component (b) defined above). The latter implies that the molar ratio of components (a) and (b) when combined is in the order of 0.14 (a) to 1 (b). However, according to the invention it is preferred that the molar ratio of components (a) and (b) is higher than 0.14 (a) to 1 (b), more preferably 0.15-0.50 (a):1 (b), even more preferably 0.20-0.40 (a):1 (b) and in particular 0.25-0.35 (a): 1 (b).

According to the invention, component (a) is preferably combined with a liquid binder. The liquid binder is used in particular for dispersing the components (a)-(c) and for enhancing the stability of the composition. Moreover, the liquid binder is used to adjust the concentrations of the active ingredients of the composition according to the invention. Obviously, the liquid binder has also additional properties, e.g. thickening properties, stabilising properties, water-binding promoting properties as is well known to the person skilled in the art. These liquid binders are preferably selected from the liquid polyols and gums and polymeric binders. Examples of suitable liquid polyols include glycerol and propylene glycol. Examples of suitable gums include natural gums and modified (semisynthetic) gums, for example acacia gum, gum arabic, caraya gum, gum tragacanth, xanthan gum and cellulose gum. Examples of suitable polymeric binders are polyvinyl pyrrolidone, casein or salts thereof, wherein the salts comprise a metal of Group 1 or Group 2 of the Periodic System. According to the invention, it is preferred that the liquid binder is glycerol, cellulose gum, polyvinyl pyrrolidone, casein or a salt thereof. According to the invention, the composition contains 1.0-80.0 percent by weight, preferably 1.5 to 75.0 percent by weight and in particular 2.0 to 70 percent by weight of at least one liquid binder, calculated on the total weight of the composition. More in particular, if the composition is intended as a skin care product, the amount of the liquid binder in the composition is preferably in the range of 10.0-25.0 percent by weight, calculated on the total weight of the composition. On the other hand, in dental care products the range is preferably 5.0 to 70.0 percent by weight, calculated on the total weight of the composition.

The inventors have also surprisingly found that in dental care products, in particular whitening products, xanthan gum has a beneficial effect on the whitening activity. According to this preferred embodiment, a dental care or whitening product according to the invention comprises xanthan gum in the range of 0.05 to 1.0 percent by weight xanthan gum, based on the total weight of the composition. Although the inventors do not wish to be bound by theory, it is believed that xanthan gum provides an improved adherence of the dental care or whitening product to enamel. A test (see Example 11) wherein commercial products were compared with the composition according to the invention demonstrated the excellent performance of the dental care or whitening product according to the invention.

However, if the composition is intended for pharmaceutical applications, the amount of the liquid binder is preferably 5.0 to 40.0 percent by weight, calculated on the total weight of the composition.

Moreover, component (a) is preferably employed as an aqueous solution comprising the binder, said aqueous solution comprising 25-75% by weight, preferably 35-65% by weight of the binder, calculated on the basis of the total weight of the aqueous solution.

Additionally, according to the invention the pH of the composition is essential for a controlled and long lasting release of the active component, i.e. oxygen. Tests have revealed that the pH is preferably in the range of 4-8, preferably 4.5-7.5 and most preferably 5.0-7.5.

The compositions according to the invention can optionally contain further 15, substances such as perfumes, aroma substances, fillers such as silica, sweeteners and flavourings, which are customarily used in pharmaceutical and cosmetic products. The compositions may also comprise additives such as hydroxyapatite and fluoroapatite which are common additives for food products such as chewing gum. However, the use of these substances can be limited to the nature of the components (a)-(d) as is well known to the person skilled in the art as well as to the intended use of the composition.

The invention also relates to a process for preparing a composition according to the invention, wherein a component (a) selected from the salts consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2, a component (b) selected from the group of oxygen donors, a component (c) selected from the group of oxygen donor stabilising agents and a component (d) selected from the group of liquid binders are combined.

The sequence by which components (a)-(d) are combined is not essential. However, according to the invention it is preferred that component (a) is first combined with component (d) and that component (b) is first combined with component (d), where after the mixtures of components (a) and (d) and components (b) and (d), respectively, are combined. Subsequently, component (c) is added.

The composition according to the invention is in particular suitable for pharmaceutical, cosmetic and food applications, in particular cosmetic skin care products such as shampoos, sunscreens and shower gels, pharmaceutical skin care products such as anti acne products, skin sprays, skin balms, skin foams, products for treatment of infections and lesions caused by the herpes simplex virus, products for treatment of athlete's foot and onychomycosis, products for the treatment of psoriasis, dermatitis, onychomycosis and itching, open wounds and burns, and products for the inhibition of hyperepithelisation, dental care products such as toothpaste, mouth conditioners, scalp sprays and whitening gels, and implant products. The composition according to the invention is also suitable for applications in self-preserving demulcent eye drops.

The composition according to the invention can also be coated on a solid carrier.

The composition according to the invention can also be used as preservation agent for pharmaceutical and cosmetic preparations.

The composition according to the present invention is in particular very suitable to be used as part of a whitening substance in combination with a flexible strip of material as disclosed in US 2002/0012685 which is incorporated by reference herein. For example, teeth can be whitened by applying a layer of the whitening substance on the flexible strip, conforming the flexible strip to the front surface of the teeth to be whitened and to the interstitial spaces between the teeth, wherein the flexible strip serves as a protection barrier for the whitening substance for a sufficient period of time for the whitening substance to act upon the teeth. Obviously, the whitening substance can be applied to the teeth to be whitened followed by the application of the flexible strip.

The composition according to the present invention can also in particular be used in dosage forms or carrier devices that provide a controlled release of the composition when the composition is applied to teeth. Such dosage forms and dosage devices are known in the art. For example, local treatment of teeth requires that a particular pharmaceutical is administered and maintained at the treatment site for a therapeutically effective period of time. The effective treatment of teeth is, however, difficult because natural bodily fluids like saliva can rapidly wash away or dilute an active compound that is applied to the teeth before the appropriate therapeutic action to the underlying surface can occur. In the mouth, saliva and the actions of eating, speaking and drinking are just some of the problems that have limited the usefulness of pharmaceutical carrier devices. Although gels and pastes have been developed as bioadhesive carriers, these types of products do not exhibit the important characteristics required for an efficient and commercially acceptable pharmaceutical delivery device for the treatment of teeth. These important characteristics include water erodability, ease of handling and application to the treatment site, comfort with minimal foreign body sensation, rapid adhesion, prolonged residence time for the protection of the treatment site and/or the delivery of a pharmaceutical. Gels are for example disclosed in U.S. Pat. No. 5,192,802, U.S. Pat. No. 5,314,915, U.S. Pat. No. 5,298,258 and U.S. Pat. No. 5,642,749, all incorporated by reference herein, but such gels have limited residence times, since bodily fluids such as saliva will quickly wash gels away from the teeth. Similarly, denture adhesive pastes which are disclosed in for example U.S. Pat. No. 4,894,232 and U.S. Pat. No. 4,518,721 have also a limited residence due to the quick dissolution by saliva. According to the invention, a preferred carrier device therefore comprises a solid stick composition comprising a water-soluble adhesive polymer, a liquid vehicle, a gelling agent, an emollient and the composition according to the present invention, wherein the solid stick composition can be applied to the teeth. The advantage of such a carrier device is that the water-soluble polymer is slowly dissolved by the bodily fluids, e.g. saliva, and that the active components of the composition of the present invention are in contact with the teeth for a prolonged time. According to the invention, the water-soluble polymers are preferably cellulose ether derivatives, preferably methyl cellulose, hydroxypropylmethylcellulose, hydroxyethylcellulose, hydroxypropylcellulose or a mixture thereof. The gelling agent is preferably an alditol derivative such as the dibenzylidene alditols. The liquid vehicle is preferably a polyhydric alcohol having 3-6 carbon atoms and 2-6 hydroxy groups, e.g. 1,2-propylene glycol, 1,3-propylene glycol, dipropylene glycol, tripropylene glycol, butylene glycol, sorbitol or a mixture thereof. The emollient may be a C₁₂-C₂₄ fatty acid ester, a diester of an organic diacid, propylene glycol diesters of short chain fatty acids, a polysiloxane or another emollient that is commonly used in the cosmetic and personal care industry. Other ingredients such as gel hardeners, fillers, colorants, preservatives, emulsifiers may be present as well in the solid stick composition.

The composition according to the invention is also suitable for the treatment of wounds and burns. If the composition according to the invention is used for this purpose, it preferably also contains an anti-oxidant. Preferred examples of anti-oxidants are sodium ascorbyl phosphate and chromanol-3.

The invention is further illustrated by the following examples which are not intended to limit the scope of protection conferred by the claims.

### EXAMPLE 1

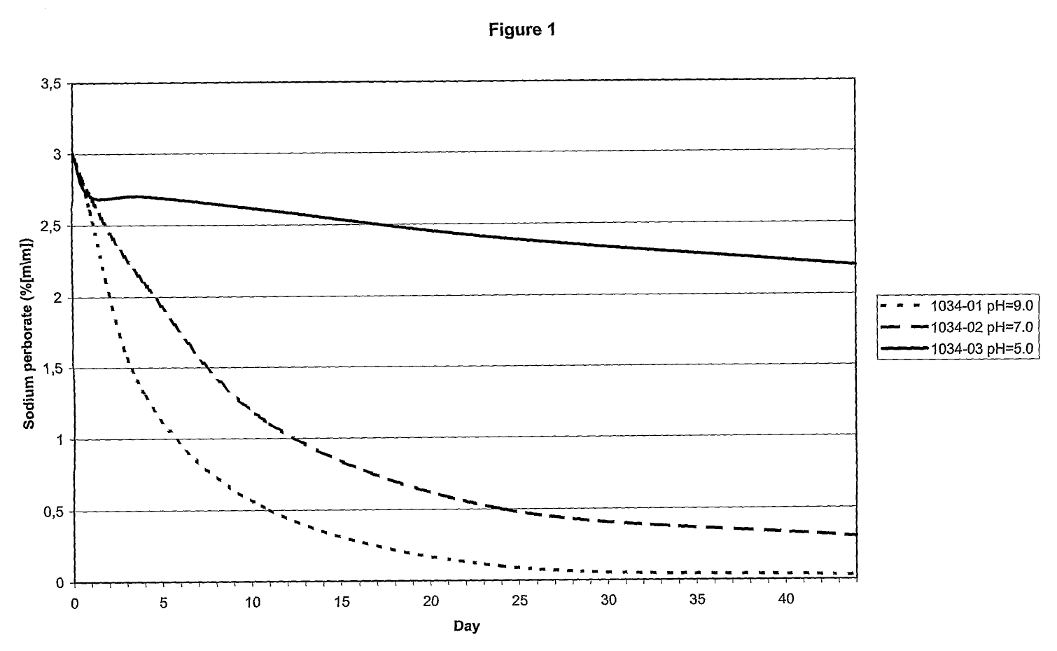

Of three compositions (see Table 1) the rate at which the content sodium perborate decreased as function of the pH was determined. The results are shown in FIG. 1.

TABLE 1

| Composition | 1034-01 | 1034-02 | 1034-03 |
|---|---|---|---|
| pH | 9.0 | 7.0 | 5.0 |
| NaBO₃•H₂O | 3.0%. by weight | 3.0%. by weight | 3.0%. by weight |
| Glycerol | 20.0% by weight | 20.0% by weight | 20.0% by weight |

The results indicate that at higher pH the content of sodium perborate decreases at a higher rate.

### EXAMPLE 2

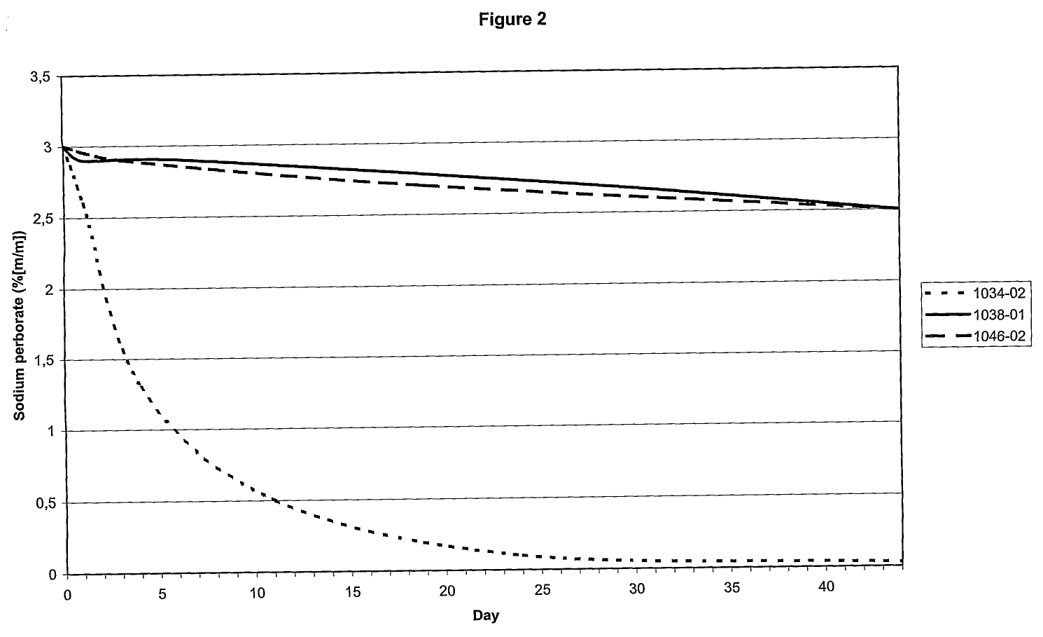

Of three compositions (see Table 2) the effect of oxygen donor stabilising agent (c) on the rate at which the content sodium perborate decreased was determined. The results are shown in FIG. 2.

The results demonstrate that the addition of component (c) has a remarkable effect on the stability of the composition, i.e. that the active component is far more slowly released. The addition of component (a) has no detrimental influence on the stabilising effect caused by component (c).

TABLE 2

| Composition | 1034-02 | 1038-01 | 1046-02 |
|---|---|---|---|
| pH | 7.0 | 7.0 | 7.0 |
| NaBO₃•H₂O | 3.0% by weight | 3.0% by weight | 3.0% by weight |
| Glycerol | 20.0% by weight | 20.0% by weight | 20.0% by weight |
| Component | — | — | 5.0% by weight |
| (a)¹ | | | |
| Component | — | 0.50% by weight | 2.0% by weight |
| (c)² | | | |

¹Aqueous solution of 50:50 (w/w) of glycerol and 4% sodium hypochlorite in water.
²Anhydrous sodium gluconate.

### EXAMPLE 3

Compositions of skin care products according to the invention are disclosed in Table 3.

TABLE 3

| Compo- | | | | | |
|---|---|---|---|---|---|
| nents | Skin care | Skin care | Skin care | Skin care | Skin care |
| (% wt.) | shampoo | shower balm | skin foam | skin balm | skin spray |
| Water | 73.55 | 66.33 | 68.96 | 77.23 | 71.96 |
| Comp. (a) | 1.65 | 1.65 | 1.65 | 1.65 | 1.65 |
| Comp. (b) | 0.29 | 0.29 | 0.29 | 0.29 | 0.29 |
| Comp. (c) | 0.50 | 0.50 | 0.50 | 0.50 | 0.50 |
| Liquid | 15.24 | 15.14 | 15.60 | 14.39 | 20.60 |
| binder | | | | | |
| Deter- | 10.67 | 15.90 | 13.00 | 0.00 | 0.00 |
| gents | | | | | |
| Polya- | 0.00 | 0.00 | 0.00 | 6.00 | 0.00 |
| crylamide | | | | | |
| Ethanol | 0.00 | 0.00 | 0.00 | 0.00 | 5.00 |

### EXAMPLE 4

Compositions of dental care products according to the invention are disclosed in Table 4.

TABLE 4

| Components | Whitening | | Mouth |
|---|---|---|---|
| (% wt.) | gel | Toothpaste | conditioner |
| Water | 47.09 | 20.10 | 88.75 |
| Comp. (a) | 2.50 | 0.86 | 0.84 |
| Comp. (b) | 2.91 | 0.14 | 0.14 |
| Comp. (c) | 2.00 | 0.50 | 0.50 |
| Liquid binder | 40.00 | 62.78 | 8.88 |
| Detergents | 0.00 | 0.47 | 0.84 |
| Silica | 5.50 | 15.00 | 0.00 |
| Ethanol | 0.00 | 0.00 | 0.00 |
| Sodium fluoride | 0.00 | 0.15 | 0.05 |

### EXAMPLE 5

Compositions for pharmaceutical applications according to the invention are disclosed in Table 5: I is an anti-acne wash lotion, II is a composition for the treatments of blisters on lips caused by the herpes simplex virus, and III is a composition for the treatment of athlete's foot and onychomycosis.

TABLE 5

| Components | | | |
|---|---|---|---|
| (% wt.) | I | II | III |
| Water | 77.65 | 45.18 | 37.16 |
| Comp. (a) | 1.66 | 27.60 | 27.60 |
| Comp. (b) | 0.29 | 5.13 | 5.13 |
| Comp. (c) | 0.50 | 2.00 | 2.00 |
| Liquid binder | 10.00 | 11.59 | 28.11 |
| Detergents | 10.00 | 0.00 | 0.00 |
| Silica | 0.00 | 8.50 | 0.00 |

### EXAMPLE 6

A gel for the treatment of burns contained the following essential components: 64.61 wt. % water, 9.20 wt. % component (a), 1.49 wt. % component (b), 2.50 wt. % component (c), 20.00 wt. % ethanol and 2.20 wt. % liquid binder. Clinical tests revealed that the gel had an unexpected favourable effect on the healing of the burns and on tissue growth.

### EXAMPLE 7

The products according to the present that are exemplified above were tested in the beta-hexosaminidase RBL 2H3 secretion assay. In this assay cells were plated such that they are close to confluent when the assay is to be carried out (in a 48-well plate, 2.5×105 cells/well; Fisher catalogue number 07-200-86). At least 3 hours was waited after plating before carrying out the experiment (up to overnight). The cells were sensitised three hours to overnight with 1.0 μg/ml IgE. Then, excess IgE was removed before stimulation and the cells were washed four times with extracellular buffer with hepes. For the stimulation, 800 ng/ml DNP-BSA suspended in extracellular buffer with 0.1% BSA was added. The total volume of the supernatant was 500 l. Incubation was subsequently carried out at 37° C. for 1 hr. In a control well, 200 nM ionomycin and 50 nM PMA for 1 hr at 37° C. were added. Following the incubation, 50 l was taken of the supernatant which was incubated with 200 l of 1 mM p-nitrophenyl N-acetyl-beta-D-glucosamine in 0.05M citrate buffer (pH 4.5) for 1 hr at 37° C. As a control for total beta-hexosaminidase concentration, cells were lysed with 1% Triton X-100 and 50 l of the supernatant was removed and incubated as described above. Both reactions were quenched following incubation by adding 500 μl of 0.05M sodium carbonate buffer (pH 10.0). The OD of each reaction was read at 405 nm. The citrate buffer was made of 49.5 ml of 0.05M citric acid and 50.5 ml of 0.05M trisodium citrate (pH 4.5). The sodium carbonate buffer was made from 60 ml Na₂CO₃ and 40 ml NaHCO₃ (pH 10.0). In this assay concentrations of the products were tested at 1.1-3.3 and 10 mg/ml. Cytotoxic effects were observed as an very high release of beta-hexosaminidase (much higher than observed with antigen stimulated cells) as a result of cell death/lysis.

### EXAMPLE 8

The products according to the invention were tested in a chemiluminescence assay. The products were cytotoxic for PMN's (polymorphonuclear neutrophils).

### EXAMPLE 9

The products according to the invention were tested in the complement assay (classical pathway and alternative pathway). Classical pathway: sheep erythrocytes coated with antibodies were contacted with human serum thereby activating the classical pathway of the complement system. The products inhibited complement activity. Alternative pathway: rabbit erythrocytes were contacted with human serum thereby activating the alternative pathway. The released amount of hemoglobin was determined by using an ELISA-reader. Inhibition of the alternative pathway of the complement system was observed.

### EXAMPLE 10

The products according to the invention were tested in the TNF-alfa assay in which human adherent mononuclear cells were activated with LPS (lipopolysaccharides). The amount of TNF-alfa that was released in this test was determined by ELISA. After the test cell death was determined by using MTT. Some stimulation was observed.

### EXAMPLE 11

A whitening composition according to the formulation disclosed for a whitening gel in Table 4 wherein the liquid binder composition comprised 0.5 percent by weight xanthan gum (based on the total weight of the composition) was evaluated in an in vitro tooth whitening test. Two commercial products, i.e. Simply White® of the Colgate-Palmolive Company (see www.colgatesimplywhite.com) and Crest® White Strips® of the Proctor & Gamble Company (see www.crest.com) were also evaluated.

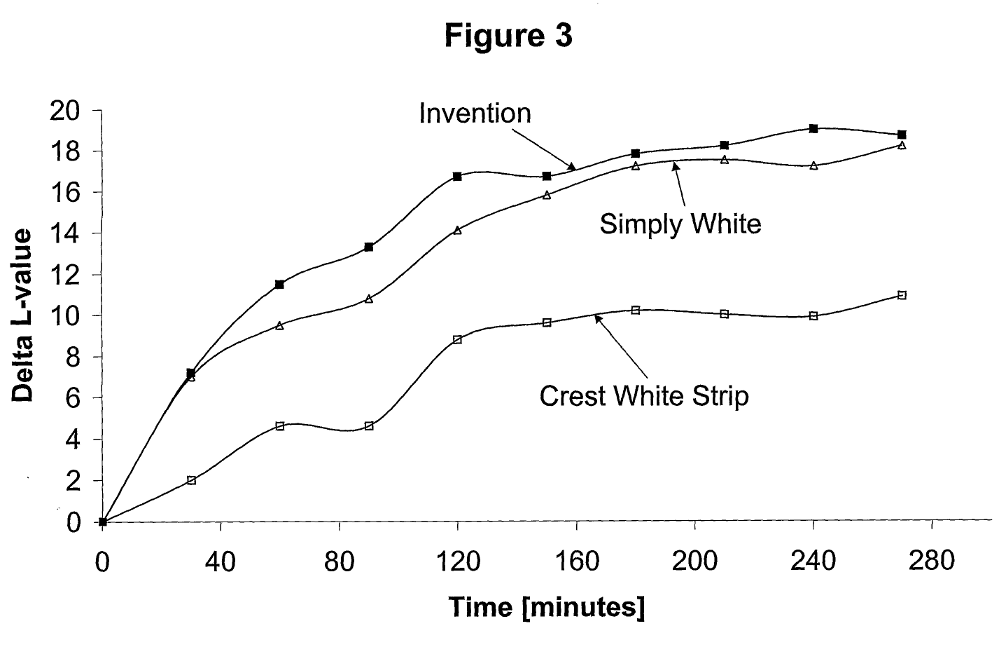

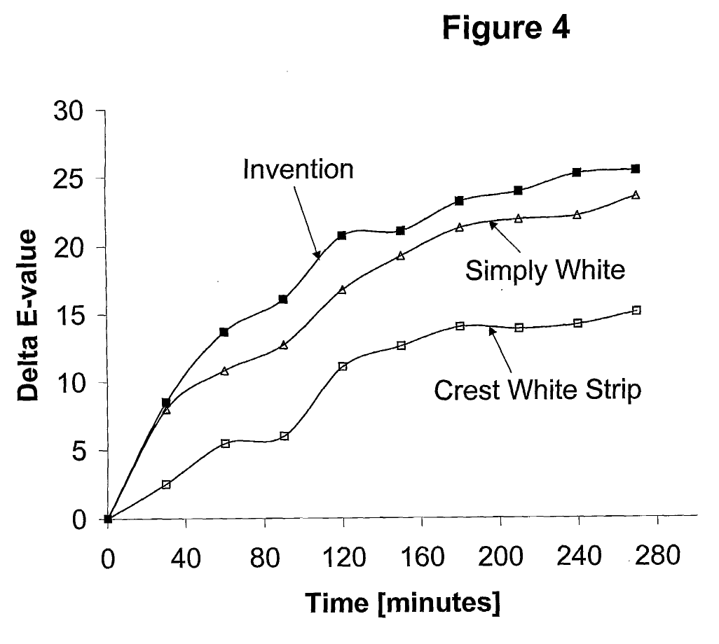

In FIGS. 3 and 4 show Delta L-value and Delta E-value, respectively, as function of time (Delta L-data are a measure for the difference between light and dark; Delta E-data is a measure for the total difference; Commission Internationale de L'Eclairage (CIE); see also E. Dybizbanski et al., M. Kacprzak et al., I Struzycka et al. and A. Kwiatkoswka et al., Research presented at the 81^{st} General Session of the IADR, Jun. 25-28, 2003). The data demonstrate that the composition according to the invention outperforms the two commercially available materials.

1-23. (canceled)

24. A stabilized, liquid, oxygen-releasing composition comprising the following components:
(a) a salt selected from the group consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor;
(c) an oxygen donor stabilizing agent; and
(d) a liquid binder.

25. The composition according to claim 24, wherein the oxygen donor is selected from the group consisting of metal perborate compounds, metal peroxide compounds, metal percarbonate compounds, metal persulfate compounds, metal perphosphate compounds, wherein the metal is an alkaline or alkaline-earth metal, halogen oxide compounds, hydrogen peroxide, and organic peroxides.

26. The composition according to claim 25, wherein the oxygen donor is a metal perborate compound, a metal percarbonate compound, a metal peroxide compound, a hydrogen peroxide, a halogen oxide compound, an organic peroxide, or a combination thereof.

27. The composition according to claim 26, wherein the metal perborate compound is a salt selected from the group consisting of cations Aₙ⁺ and anions derived from borates according to the general formula [BₚO_{q}]^{r} wherein A is a metal selected from Groups 1 or 2 of the Periodic System of the Elements, p=1-4, q=1-8, and r=1-3.

28. The composition according to claim 26, wherein the metal peroxide compound is calcium peroxide, magnesium peroxide, or both.

29. The composition according to claim 26, wherein the halogen oxide compound is chlorine oxide.

30. The composition according to claim 27, wherein p=1, q=2 or 3 and r=1.

31. The composition according to claim 27, wherein p=1, q=3 and r=1.

32. The composition according to claim 24, wherein the oxygen donor stabilizing agent is an organic acid, a salt thereof, a saccharide, or a combination thereof.

33. The composition according to claim 32, wherein the salt of an organic acid is an inorganic salt.

34. The composition according to claim 32, wherein cations of the salts are metals selected from Groups 1 or 2 of the Periodic System of Elements.

35. The composition according to claim 32, wherein the organic acid is a chelating organic acid.

36. The composition according to claim 32, wherein the organic acid is a carboxylic acid having one or more hydroxy and/or ammo groups; a polycarboxylic acid, optionally having one or more hydroxy and/or amino groups; or both.

37. The composition according to claim 36, wherein the polycarboxylic acid is a hydroxypolycarboxylic acid or a aminopolycarboxylic acid.

38. The composition according to claim 24, wherein the molar ratio of component (b) to component (c) is 0.1-5.0 (b) to 1.0 (c).

39. The composition according to claim 24, wherein the molar ratio of component (a) to component (b) is higher than 0.14 (a) to 1.0 (b).

40. The composition according to claim 24, wherein the composition has a pH in the range of 4-8.

41. The composition according to claim 24, wherein the liquid binder is a liquid polyol, a gum, or both.

42. The composition according to claim 41, wherein the liquid polyol is glycerol.

43. The composition according to claim 41, wherein the gum is a cellulose gum.

44. The composition according to claim 24, wherein the composition comprises 1.0 to 80.0 percent by weight of at least one liquid binder.

45. The composition according to claim 24, wherein m=1.

46. A process for preparing a composition according to claim 24, comprising combining, in any order, the following components:
(a) a salt selected from the group consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor;
(c) an oxygen donor stabilizing agent; and
(d) a liquid binder.

47. A pharmaceutical or cosmetic preparation comprising the following components:
(a) a salt selected from the group consisting of cations Aₙ⁺ and anions derived from halogen oxides according to the general formula [OₘX]⁻, wherein A is a metal selected from Groups 1 or 2 of the Periodic System of the Elements, X is a halogen atom, m=1-4, n=1 or 2;
(b) an oxygen donor;
(c) an oxygen donor stabilizing agent; and
(d) a liquid binder.
